(19) United States
(12) Patent Application Publication
(10) Pub. No.: US 2019/0000696 A1
(43) Pub. Date: Jan. 3, 2019

(54) SHORT FIBER NONWOVEN FABRIC, LOOP MEMBER FOR SURFACE FASTENER, AND SANITARY ARTICLE

(71) Applicant: 3M INNOVATIVE PROPERTIES COMPANY, St Paul, MN (US)

(72) Inventor: Kenichiro Morishita, Tokyo (JP)

(21) Appl. No.: 16/064,469

(22) PCT Filed: Dec. 15, 2016
(86) PCT No.: PCT/US2016/066809

§ 371 Date: Jun. 21, 2018

(30) Foreign Application Priority Data
Dec. 25, 2015 (JP) 2015-253849

(51) Int. Cl. A61F 13/56; D04H 1/541; D04H 1/559; D04H 11/08; B32B 5/02; B32B 5/26

(52) U.S. Cl. CPC A61F 13/5633; B32B 2262/0284; D04H 1/559; D04H 11/08; B32B 5/022; B32B 5/26; D10B 2509/026; D10B 2321/022; D10B 2321/021; B32B 2250/20; B32B 2255/02; B32B 2555/02; B32B 2307/724; B32B 2262/0253; B32B 2262/12; D10B 2331/04; D04H 1/541

(57) ABSTRACT

One of challenges to be overcome is providing short-fiber nonwoven and loop component with appropriate balance of air-permeability and softness. One of solutions is loop component for mechanical fastener including top layer comprising short-fiber nonwoven and substrate layer comprising short-fiber nonwoven, wherein ratio [average fineness (denier) of the top layer] against [average fineness (denier) of the substrate layer] is 1.5-30, softness of the substrate is less than 60 mm in MD direction and less than 50 mm in CD direction by 45 degree cantilever method, and air permeability of the substrate is 10 to 100 cm3/sec*cm2 by Frajour type method.

### TECHNICAL FIELD

The present invention relates to a short fiber nonwoven fabric, a loop member for a surface fastener, and a sanitary article.

### BACKGROUND ART

Conventionally, surface fasteners have been widely used to secure or bind a variety of articles including fiber products, plastic products, paper products, industrial articles, electronic components, building materials, and the like. For example, sanitary articles (e.g. paper diapers, and the like) on which surface fasteners are attached as fastening members are known. Surface fasteners with a variety of engaging methods are known, such as, for example, paired fasteners constituted from a male member that has a hook-like engaging element and a female member capable of engaging with the engaging element. Among these, surface fastener-use fastening members using nonwoven fabric are advantageous in that they have pliability and air-permeability and, thus, conventionally, many types have been proposed.

For example, Patent Document 1 describes a loop material for a hook and loop type fastener constituted of a composite nonwoven fabric. The composite nonwoven fabric consists of a loop layer of a carded nonwoven fabric of thermoplastic crimped staple fiber, the staple fiber being from 1.5 to 6.0 dTEX, and said carded nonwoven fabric having a basis weight of from 10 to 35 g/m²; a backing layer of a spunbond or spunmelt nonwoven fabric having a basis weight of 5 to 30 g/m², the loop layer being superimposed face-to-face with the backing layer; and a plurality of bond regions joining the loop layer to the backing layer and rendering the bond regions substantially air impermeable, the bond regions comprising between 35 to 55% of a surface area of the loop material.

Patent Document 2 describes a female member for a face fastener, comprising a web which includes a heat-melt-adhering composite fiber body; a plurality of entangled loops formed in a first surface of the web; and a densified heat-melt-adhered layer formed in a second surface of the web. The web includes fibers having a fineness of about 0.5 to 10 deniers and a tensile strength of greater than about 2 g/denier, and the second surface is more dense than the first surface so that the plurality of entangled loops formed in the first surface can be forcibly engaged with elements formed on a surface of a male member, with a peel strength required to separate the plurality of entangled loops from the elements formed on the surface of the male member being at least 20 gf/cm.

Patent Document 3 describes a process for forming a liquid-impermeable, breathable sheet having a fibrous surface. The process comprises the steps of forming a sheet having a first fibrous surface and a second fibrous surface; subjecting the sheet to pressure and a z-gradient temperature differential sufficient to melt the fibers of the first surface and form the melt into a liquid impermeable, non-breathable skin without significantly altering the fibers of the second surface; depositing fibers upon the skin while the skin is at least semi-molten to form a fibrous/skin/fibrous material; and aperturing the liquid impermeable, non-breathable skin to make it breathable, while the skin remains liquid-impermeable.

### PATENT LITERATURE

Patent Document 1: WO/2008/130807

Patent document 2: Specification of U.S. Pat. No. 5,786,060

Patent document 3: Specification of U.S. Pat. No. 5,470,424

### SUMMARY OF INVENTION

However, with, for example, adult diapers, in some cases, a mode of use is adopted in which a diaper is used in combination with a pad, disposed inside the diaper and, while the inside pad is changed frequently, the outside diaper is only changed once every three days or so (or once every 20 changes of the pad, or so). Thus, the diaper is used for a comparatively long and continuous period of time. In this case, there is a need for the surface fasteners of the diaper to retain excellent engagement strength (particularly peel strength and shear strength) between the fastening members, even after multiple repetitions (e.g. about 20 times) of detaching and attaching between the paired fastening members (i.e. retention of engagement strength when subjected to repeated detaching and attaching). However, with loop members for which the engaging element is nonwoven fabric (and particularly short fiber nonwoven fabric), the fibers constituting the nonwoven fabric become prone to shedding or failing due to repeated detaching and attaching and, as a result, it is difficult to achieve retention of the engagement strength in cases of repeated detaching and attaching. On the other hand, knit loops generally have excellent retention of engagement strength when subjected to repeated detaching and attaching, but air permeability and pliability are low. As such, there is a need for a fastening member in which nonwoven fabric is used to ensure excellent air permeability and pliability and, at the same time, also has excellent retention of engagement strength when subjected to repeated detaching and attaching.

Additionally, in articles that include surface fasteners (e.g. sanitary articles such as diapers, and the like), surface fastener-use fastening members that have a print layer are sometimes used. Examples of such a surface fastener-use fastening member include those in which a print layer is formed on one main surface of a nonwoven fabric and the other main surface of the nonwoven fabric is configured as an engaging surface for engaging with another surface fastener-use fastening member. In such a configuration, a design provided on the print layer is visible via the nonwoven fabric. By securing the main surface of the print layer side of the surface fastener-use fastening member to a body portion of an article, an article including a surface fastener with a design can be formed. However, conventionally, print layers provided on nonwoven fabrics do not necessarily have sufficient sharpness. Additionally, further reductions in the cost of fastening members are desired.

Moreover, as described above, while nonwoven fabric has higher air permeability and pliability than knit loops, there are problems in that if the air permeability is excessively high, handling at the time of production declines, and if the air permeability is lowered, pliability will also decline.

An object of the present invention is to solve the problems described above and provide a nonwoven fabric and a loop member that have an appropriate balance of air permeability and pliability. This objective is shared for both adults and children, but from the perspective of pliability, the need for a solution is particularly high for children. In addition to this balance, an object of the present invention is to provide a loop member that is producible at low cost and has excellent engagement strength between the paired fastening members, excellent retention when subjected to repeated detaching and attaching, and excellent print characteristics (sharpness of print layer). On this objective, from the perspective of durability, the need for a solution is particularly high for adults.

### Solution to Problem

One aspect of the present invention provides a loop member for surface fastener comprising:

a loop layer of short fiber nonwoven fabric; and

a backing layer of short fiber nonwoven fabric; wherein

a ratio of an average fineness of the fiber in the loop layer to an average fineness of the fiber in the backing layer (average fineness of fiber in the loop layer/average fineness of fiber in the backing layer) is from 1.5 to 30;

a degree of pliability of the loop member when measured by a Cantilever method is 60 mm or less in a machine direction and 50 mm or less in a cross direction; and

an air permeance of the loop member when measured by a Frazier method is from 10 to 100 cm³/s×cm².

Another aspect of the present invention provides a short fiber nonwoven fabric wherein:

a degree of pliability measured by a Cantilever method is 40 mm or less in a machine direction and 30 mm or less in a cross direction; and

an air permeance measured by a Frazier method is 150 cm³/s×cm² or less.

### Advantageous Effects of Invention

According to the present invention, a nonwoven fabric, a loop member for a surface fastener, and a sanitary article can be provided that have an appropriate balance of air permeability and pliability.

### BRIEF DESCRIPTION OF DRAWING

FIG. 1 is a drawing illustrating an example of a loop member according to an aspect of the present invention.

FIG. 2 is a drawing illustrating an example of a loop member according to an aspect of the present invention, and depicts a state in which a loop layer and a backing layer are secured by embossing.

FIG. 3 is a figure showing a surface state image of the loop layer of Working Example 1.

FIG. 4 is a figure showing a surface state image of the loop layer of Comparative Example 1.

### DESCRIPTION OF EMBODIMENT

An exemplary aspect of the present invention will be described hereafter, but the present invention is not limited to the following aspect, and various modifications within the spirit and scope of the claims are comprehended in the present invention. Unless otherwise noted, each characteristic value in the present disclosure is intended to be a value measured with the method described in the Examples section of the present disclosure or a method that would be understood to be equivalent thereto by a person having ordinary skill in the art.

A loop member of the present disclosure can be used as a loop member for forming a surface fastener with various conventionally known fastening means. In one aspect, the loop member of the present disclosure can be used as a female member, and can be combined with a male member to constitute a surface fastener. In another aspect, the surface fastener may be a pair of members in which, for example, both the structure of the male member and the loop member of the present disclosure, namely the female member, exist on the same surface. The loop member of the present disclosure may, for example, directly engage with a wall, fabric, or the like.

More specifically, with the loop member of the present disclosure, it is intended that the short fiber nonwoven fabric included in the loop layer functions as an engaging element, and the short fiber nonwoven fabric included in the backing layer constitutes a print surface. A hook is an example of a preferable male member as it is capable of strong engagement with the nonwoven fabric engaging element. The hook is constituted of a protrusion, protruding in a thickness direction of the surface fastener. The protrusion may be of any type as long as satisfactory engaging force can be obtained, but, for example, a mushroom-shape, an anchor-shape, or a J-shape is preferable. Pin density is generally about from 500 to 5,000 pins per square inch. The material may be selected from polypropylene, polyester, polyethylene, polyamide, and copolymers or mixtures thereof. The loop member of the present disclosure may be a portion of a fastening member configured such that the hook and the loop member, which includes the short fiber nonwoven fabric as the engaging element with which the hook is engageable, both exist on the same surface.

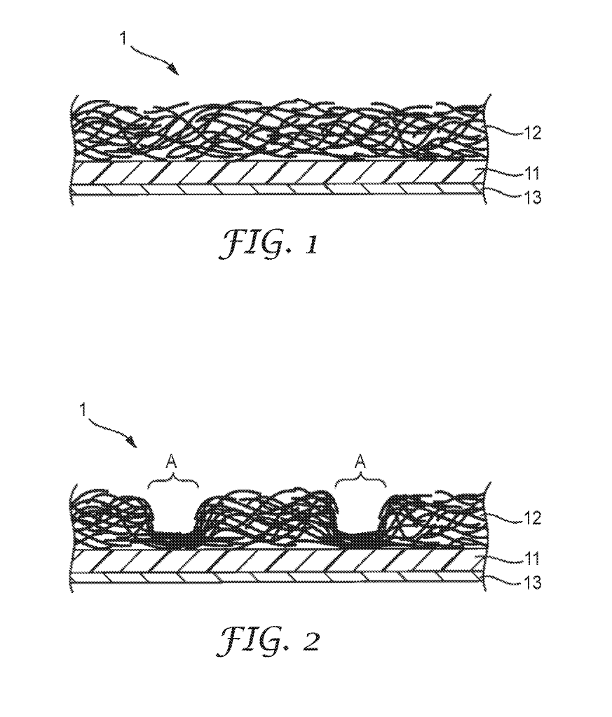

FIG. 1 is a drawing illustrating an example of the loop member according to an aspect of the present invention. As illustrated in FIG. 1, a loop member 1 includes a backing layer 11 and a loop layer 12. In a typical aspect, the loop member 1 may further include a print layer 13.

The loop layer and the backing layer include short fiber nonwoven fabric. In a typical aspect, the loop member may be essentially constituted of nonwoven fabric and, more typically may be essentially constituted of short fiber nonwoven fabric. Loop members essentially constituted of nonwoven fabric can have pliability and air permeability of a degree suitable for use as sanitary articles. Short fiber nonwoven fabric is advantageous because a thin and soft layer can be formed therefrom. Note that, in the present disclosure, the term “short fiber nonwoven fabric” refers to nonwoven fabric for which at least a main portion (more than 50 mass % of the constituent fiber) is constituted of staple (i.e. short fiber), and is distinguished from nonwoven fabric constituted of filament (i.e. long fiber). Short fiber nonwoven fabrics encompass carded nonwoven fabrics, air-laid nonwoven fabrics, air-laid nonwoven fabrics, and the like. On the other hand, long fiber nonwoven fabric generally encompasses spunbond nonwoven fabrics and the like. While not limited hereto, the staple generally may have a fiber length of a few hundred millimeters or less.

The loop layer includes fused short fiber nonwoven fabric. In the present disclosure, the term “fused short fiber nonwoven fabric” refers to short fiber nonwoven fabric that has a form in which the fibers constituting the short fiber nonwoven fabric are fixed together as a result of the fibers being melted. In performing state observation (e.g. state observation using an optical microscope), when melting marks of fiber material are found on the fiber surface of the short fiber nonwoven fabric, and the fibers are bonded together at the sites of the melting marks, it can be confirmed that the short fiber nonwoven fabric is fused. Here, the term “melting marks” refers to marks left by performing treatment purposed to fuse only the loop layer (as such, the marks are only seen in the loop layer), and are distinguished from, for example, melting marks formed as a result of treatment for other purposes such as bonding the loop layer to the backing layer or the like (such melting marks are seen throughout both the loop layer and the backing layer). Additionally, the fusing can be confirmed by the short fiber nonwoven fabric having a slightly rigid surface when touched with a hand. In the present disclosure, the fusing to form the fused short fiber nonwoven fabric can be achieved through high temperature air-through processing. In the present disclosure, the term “high-temperature air-through processing” of the short fiber nonwoven fabric refers to processing in which air of high-temperature (at least higher than or equal to the melting point of the material on the outside of the fibers of the short fiber nonwoven fabric) is passed through the short fiber nonwoven fabric in a thickness direction thereof. As another method for the fusing, a technique by which air of high-temperature is not passed through and, instead, the outside of the fibers is melted by heating means or chemically in order to fix the fibers together can be considered. However, from the perspective of fusing not only the surface of the short fiber nonwoven fabric, but also the (outside of the fibers on the) inner side, the high-temperature air-through processing is preferable. Additionally, in diapers and similar sanitary article applications, the use of chemicals and the like to melt the fibers is frequently avoided and, thus, the high-temperature air-through processing is preferable. Thus, the fibers are fused through a method other than methods that include applying a large amount of pressure directly to the short fiber nonwoven fabric, such as rolling and the like. As a result, it is possible to improve durability while maintaining the engaging force of the loop member.

The backing layer includes calendered short fiber nonwoven fabric. In the present disclosure, the term “calendered short fiber nonwoven fabric” refers to short fiber nonwoven fabric that has a surface form that has been smoothed by the application of pressure. Accordingly, in the present disclosure, the calendering for forming the calendered short fiber nonwoven fabric may encompass processing in which a layer to be processed is passed between a pair of smoothing rolls and also, for example, processing in which a layer to be processed is smoothed by being passed between a smooth roll and an uneven roll (e.g. a thermal bond roll) and the like. Note that in cases where using an uneven roll, for example, it is intended that a person skilled in the art will perform appropriate adjustments of the process in order to obtain the desired smoothing results (e.g. performing the operation of passing the layer to be processed between the rolls a plurality of times, adjusting the point-bonding conditions so that the processing area in the point-bonding (described later) is comparatively larger, and the like). Note that the “high-temperature air-through processing” and the “calendering” are different in that while the latter includes bringing the short fiber nonwoven fabric direction into contact with a roll or the like for the purpose of smoothing, the former does not.

With the loop member of the present disclosure, the backing layer and the loop member are separate layers and, thus, the characteristics of each layer can be independently controlled depending on the purpose thereof. The present inventor focused on the need to provide a print surface with high smoothness in order to realize excellent print characteristics (specifically, the realization of a sharply printed design). The present inventor also focused on the need to ensure engagement strength (particularly peel strength and shear strength) between a pair of fastening members (e.g. a male member when using the loop member of the present disclosure as the female member; also referred to as a “hook”), and the need to ensure retention of the engagement strength when subjected to repeated detaching and attaching. Additionally, conventionally, nonwoven fabrics are point-bonded and, as such, the present inventor did not focus on the print surface being smooth, but rather focused on the facts that ink drops exist unstably on the print surface, the loop member is thick, and that when viewing the print layer through the loop member, the printed image appears blurry as the main causes for the viewed printed image being indistinct. The present inventor also focused on the fact that conventional loop members in which nonwoven fabric is used do not have engagement strength retention that can withstand repeated detaching and attaching.

Moreover, the present inventor investigated laminating a loop layer that includes a nonwoven fabric that is fused and has larger fineness compared to the nonwoven fabric used in the backing layer, and a backing layer that has been thinly smoothed by calendering; and, in so, discovered that the engagement strength between the fastening members and engagement strength retention when subjected to repeated detaching and attaching, and the print characteristics and shape retention as a member can both be achieved at high levels. Additionally, thin loop members (that is, the product obtained by laminating the loop member and the backing layer) are advantageous in that they are low cost and have excellent pliability.

The loop layer may function as the engaging element. In one aspect, the short fiber nonwoven fabric included in the loop layer is engageable with a hook. Various types of thermoplastic resins may be used as the material of the hook, and examples thereof include polyethylene (e.g., high density polyethylene), polypropylene, polystyrene, polyvinyl chloride, polyethylene terephthalate, polybutylene terephthalate, nylon, polycarbonate, polymethyl methacrylate, polyacetal, polymethylpentene, acrylonitrile-styrene-butadiene, polyphenylene ether, polyphenylene sulfide, as well as styrene-butadiene-styrene, styrene-isoprene-styrene, and similar styrene-based elastomers, ethylene-α-olefin copolymer and similar olefin-based elastomers, ester-based elastomers, amide-based elastomers, urethane-based elastomers, vinyl chloride-based elastomers, silicone-based elastomers, fluorine-based elastomers, alloys thereof, and the like.

Examples of the fibers constituting the short fiber nonwoven fabrics included in each of the loop layer and the backing layer include polyolefins (e.g. polyethylene, polypropylene, and the like), polyester (e.g. PET, PBT, and the like), polyamide, polyurethane, EVA (ethylene-vinyl acetate), polylactic acid, rayon, fibers of copolymers and mixtures thereof, natural fibers, and the like.

In an aspect, from the perspective of preventing damage to the loop layer (the fiber falling out, or the like) due to engagement with the other fastening member, high-strength polyamide may be used for the loop layer. On the other hand, in consideration of material costs and environmental safety, polyethylene, polypropylene, polyester and the like are preferably used in the loop layer and/or the backing layer.

The fibers constituting the short fiber nonwoven fabric may be hydrophilic or hydrophobic. The fibers may also be composite fibers. Examples of preferable fiber forms of the composite fiber include sheath and core type (concentric type and eccentric type), parallel type (side-by-side type), split type (e.g. the cross-section is split into arc forms), and the like. Additionally, the fibers may be modified cross-section fibers, crimped fibers, heat-shrinkable fibers, or the like. These fibers may be used singly or in combinations of two or more kinds.

Additional examples include a two-component-type elastic composite fiber having a hard elastic component including crystalline polypropylene as a first component and having a thermoplastic elastomer as a second component; and mixed fibers including the two-component-type elastic composite fiber and other fibers.

In a preferred aspect, the fibers constituting the short fiber nonwoven fabric included in the loop layer have a core-in-sheath structure. Additionally in a preferred aspect, the fibers constituting the short fiber nonwoven fabrics included in each of the loop layer and the backing layer have a core-in-sheath structure. Core-in-sheath fibers that have a core with a first melting point (e.g. polyamide) and a sheath with a second melting point lower than the first melting point (e.g. polyethylene) are advantageous as the fibers that have a core-in-sheath structure because heat fusibility thereof is excellent. In consideration of material costs and environmental safety, and from the perspective that polyethylene, polypropylene, polyester and the like are preferably used in the loop layer and/or the backing layer, examples of the core-in-sheath fibers that have a core with a first melting point and a sheath with a second melting point lower than the first melting point include core-in-sheath fibers that have a polypropylene core and a polyethylene sheath, core-in-sheath fibers that have a polypropylene core and a modified polyethylene sheath, core-in-sheath fibers that have a polypropylene core and a modified polypropylene sheath, and the like. In an aspect, from the perspectives of being light-weight, having high strength, having high pliability, and the like, core-in-sheath fibers that have a polypropylene core and a polyethylene sheath may be selected. Particularly, from the perspective of achieving excellent formation of the fusing sites, it is advantageous that the loop layer includes the short fiber nonwoven fabric constituted of fibers that have the core-in-sheath structure described above.

For example, in a preferable aspect of a case where the fibers constituting the short fiber nonwoven fabric included in the loop layer are the core-in-sheath fibers that have a core with a first melting point and a sheath with a second melting point lower than the first melting point, from the perspective of obtaining excellent mechanical strength of the loop layer, the first melting point is about 150° C. or higher, about 160° C. or higher, or about 170° C. or higher and, in cases where polyethylene or polypropylene is used, is about 200° C. or lower in light of the properties of the material, and in cases where polyester is used, is about 300° C. or lower in light of the properties of the material. Additionally, in a preferable aspect, from the perspective of obtaining excellent mechanical strength of the loop layer, the perspective of achieving excellent formation of the fixing sites by fusing in the loop layer, and the perspective of obtaining excellent pliability of the loop layer, the second melting point is about 130° C. or lower, about 120° C. or lower, or about 110° C. or lower and, from the perspective that polymeric material is used, is about 80° C. or higher or about 100° C. or higher. A combination of the first melting point in the range described above and the second melting point in the range described above is particularly preferable. Note that, from the perspective of forming a short fiber nonwoven fabric with excellent air permeability and smoothness, the first melting point and the second melting point being the ranges described above, and combinations thereof are advantageous for the backing layer as well. Additionally, both cases where the core has a plurality of melting points and cases where the sheath has a plurality of melting points are possible. In these cases, all of the plurality of melting points of the core are the first melting point and all of the plurality of melting points of the sheath are the second melting point, but the lowest of each particularly contributes to the fusing and shape retention. In an aspect, an example of the first melting point of the core and the second melting point of the sheath in the present disclosure may be related to the lowest melting point of the first melting point of the core and the lowest melting point of the second melting point of the sheath. Note that the melting points are values measured by DSC or the like.

In another preferred aspect, the fibers constituting the short fiber nonwoven fabric included in the loop layer may be fibers of a single material that has a melting point within the range described above as, for example, the first melting point.

In a preferred aspect, an average fineness of the loop layer is about 2.0 denier or greater and about 15.0 denier or less. From the perspective of obtaining excellent engagement strength, the average fineness is preferably about 2.0 denier or greater or about 4.0 denier or greater and, from the perspective of maintaining excellent pliability of the loop member, is preferably about 15.0 denier or less, about 12.0 denier or less, about 10.0 denier or less, about 8.0 denier or less, or about 6.0 denier or less.

In a preferred aspect, an average fineness of the backing layer is about 0.5 denier or greater and about 3.0 denier or less. From the perspective of retaining the mechanical strength and ease of production of the backing layer, the average fineness is preferably about 0.5 denier or greater, about 0.7 denier or greater, about 0.9 denier or greater, or about 1.0 denier or greater and, from the perspective of providing a smooth print surface and imparting excellent print characteristics, is preferably about 3.0 denier or less, about 2.5 denier or less, about 2.0 denier or less, or about 1.5 denier or less.

A ratio of the average fineness of the fibers in the loop layer to the average fineness of the fibers in the backing layer (average fineness of fibers in loop layer/average fineness of fibers in backing layer) is about 1.5 or greater and about 30 or less. From the perspective of obtaining both the engagement strength of the loop layer and the print characteristics of the backing layer, the ratio is about 1.5 or greater, and preferably is about 1.7 or greater or about 2 or greater. Additionally, from the perspectives of maintaining the pliability of the loop member, maintaining the mechanical strength of the backing layer, and ensuring the ease of production, the ratio is about 30 or less, and preferably is about 18 or less or about 6 or less.

This ratio is calculated from values obtained by measuring the average fineness of each of the loop layer and the backing layer using a fineness measuring device, namely Vibromat ME (manufactured by Textechno) or an equivalent measuring device. Specifically, first, about 10 strands of fiber are collected in as random a manner as possible. When collecting, broken fibers and otherwise damaged fibers cannot be used in the measuring, so such fibers are not collected. From the fibers that are collected, the end of one fiber is grabbed using tweezers and, without twisting or stretching the fiber, both ends of the fiber are secured in the clamp of the measuring instrument. Here, the thread is disposed so as to be vertical. At a set fiber length and tension, the fixed oscillation rate of the fiber secured in the clamp is measured from the oscillations thereof and converted to fineness. This operation is repeated five times, and the fineness average value of five strands of fiber is defined as the average fineness. The measurement environment is set to a temperature of 21° C.±1° C. and a humidity of 65%±2%. The ratio described above (average fineness of fibers in loop layer/average fineness of fibers in backing layer) is calculated from the calculated average fineness of each of the loop layer and the backing layer.

Note that in cases where a plurality of types of fibers is used in a layer of the loop layer or the backing layer, the average fineness of this layer is the weight ratio weighted average fineness of the plurality of types of fibers. For example, in cases where a fiber A and a fiber B are used at a predetermined weight ratio in the loop layer, the average fineness of this loop layer is calculated by: the average fineness found for fiber A×the weight ratio of fiber A+the average fineness found for fiber B×the weight ratio of fiber B.

The short fiber nonwoven fabrics included in each of the loop layer and the backing layer may be short fiber nonwoven fabrics produced using a common method for producing short fiber nonwoven fabric and, examples thereof include carded nonwoven fabrics, air-laid nonwoven fabrics, and the like. Here, in cases where long fiber nonwoven fabric produced by a spunbond method or a meltblown method is used as the backing layer, it is necessary to use a high density nonwoven fabric in order to reduce the air permeability of the resulting backing layer. Additionally, in cases where the backing layer constituted of this long fiber nonwoven fabric is made thinner through calendering, reduced thicknesses will lead to greater tendencies for the resulting backing layer to become rigid. On the other hand, in cases where the backing layer including the short fiber nonwoven fabric is made thinner through calendering, it is easier to impart pliability and an appropriate degree of air permeability. The method of bonding the fibers of the short fiber nonwoven fabric may be thermal bonding, chemical bonding, hydroentangling, needle punching, stitch bonding, steam jetting, or the like. In a preferred aspect, from the perspective of obtaining a thin layer with excellent pliability, the short fiber nonwoven fabric included in the backing layer is a thermally bonded nonwoven fabric. In a preferred aspect, the short fiber nonwoven fabric included in the loop layer is preferably obtained by fusing the short fiber nonwoven fabric via high-temperature air-through processing.

In the fused short fiber nonwoven fabric included in the loop layer, the fibers constituting the short fiber nonwoven fabric are bonded together at fixing sites due to melting of the fibers (typically melting of the surface of the fibers). As a result, the short fiber nonwoven fabric of the loop layer has excellent mechanical strength and, as such, can display excellent retention of engagement strength when subjected to repeated detaching and attaching.

The fusing can be performed using any device capable of high-temperature air-through processing. For example, an oven used in the production of the nonwoven fabric can be used as the device. However, in a typical aspect of the present disclosure, the fusing is performed at a temperature higher than normal temperature conditions set for each of the nonwoven fabric materials when producing the nonwoven fabrics. Accordingly, in a typical aspect of the present disclosure, a device capable of air-through processing at a temperature such as that described later is used.

When fusing the short fiber nonwoven fabric of the aforementioned material, a higher degree of melting of the fibers, brought about by setting the melt temperature to a higher temperature or the like, contributes to an increase in the fixing sites between the fibers, formed by the melting of the fiber surface. When there are many fixing sites between the fibers, gaps between the fibers in the short fiber nonwoven fabric will decrease, and the volume of the short fiber nonwoven fabric will decrease. In a typical aspect, in cases where the fusing is performed by high-temperature air-through processing, the fusing temperature (specifically the air temperature) and the air blow amount can be set in accordance with the desired degree of formation of the fixing sites, according to the purpose. Hereinafter, an aspect of an example of the temperature and the air blow amount is described, but the present disclosure is not limited thereto.

In a preferred aspect in which the sheath is polyethylene, the short fiber nonwoven fabric of the loop layer is fused at a fusing temperature (e.g. the air temperature in the high-temperature air-through processing) of about 135° C. to about 160° C. From the perspectives of ensuring excellent formation of the fixing sites and, thereby, preventing fiber shedding of the short fiber nonwoven fabric of the loop layer, obtaining excellent mechanical strength, and obtaining excellent retention of the engagement strength, the fusing temperature is set to about 135° C. or higher or about 140° C. or higher and, from the perspective of maintaining excellent pliability and air permeability of the loop layer, is set to about 160° C. or lower, about 150° C. or lower, or about 145° C. or lower.

In a preferred aspect, from the perspective of obtaining excellent formation of the fixing sites, a difference (T1−T2) between a fusing temperature (T1) and a melting point (T2) of the material constituting the surface of the fibers to be crimped is about 5° C. or greater, about 10° C. or greater, or about 30° C. or greater.

In a preferred aspect, from the perspectives of ensuring excellent formation of the fixing sites and, thereby, preventing fiber shedding of the short fiber nonwoven fabric of the loop layer, obtaining excellent mechanical strength, and obtaining excellent retention of the engagement strength, the air blow amount in the high-temperature air-through processing is about 1% or greater, about 10% or greater, about 20% or greater, or about 30% or greater and, from the perspective of preventing the fixing sites from becoming excessively numerous and maintaining excellent pliability and air permeability, is about 100% or less or about 50% or less. Note that the air blow amount is selected so as to be balanced with the temperature.

In a preferred aspect, a tensile strength at break of the loop layer is about 20 N/50 mm or greater and about 200 N/50 mm or less. In a preferred aspect, from the perspective of obtaining excellent engagement strength of the loop member, the tensile strength is about 20 N/50 mm or greater, about 25 N/50 mm or greater, or about 30 N/50 mm or greater and, from the perspective of obtaining excellent pliability and air permeability of the loop member, is about 200 N/50 mm or less or about 100 N/50 mm or less.

In a typical aspect, the loop layer is substantially not subjected to calendering for the purpose of surface smoothing. As a result, the short fiber nonwoven fabric included in the loop layer can be caused to function excellently as the engaging element. Note that compression applied to the loop layer such as compression from the rolls or the like when bonding/fixing the loop layer and the backing layer is not eliminated. For example, in cases where only one surface of the short fiber nonwoven fabric layer is subjected to surface smoothing, sometimes a portion of the other surface of the short fiber nonwoven fabric layer is subjected to surface smoothing at the same time. In these cases, there is a possibility that the engagement strength (e.g. peel strength and shear strength) of the short fiber nonwoven fabric present on the other surface will decline. In contrast, with the loop member of the present disclosure, the loop layer is combined with the pre-calendered backing layer and, as such, the loop layer is not affected by the calendering of the backing layer. Therefore, the loop layer can be formed without compromising the desired engagement strength.

The method and conditions of the calendering for obtaining the calendered short fiber nonwoven fabric included in the backing layer can be set according to the purpose thereof. If the density of the backing layer increases due to the calendering, the backing layer can have a smooth surface.

For example, the calendering is performed under conditions set so that the short fiber nonwoven fabric for use in the backing layer is compressed to a desired thickness. In an illustrated aspect, in cases where the backing layer is constituted of polypropylene/polyethylene core-in-sheath fibers, the calendering can be performed under conditions of, for example, a roll temperature of about 120° C. to about 180° C.

The short fiber nonwoven fabric of the calendered backing layer is beneficial in that it contributes to the stable bonding of the backing layer with the loop layer, that is, to the stable retention of the loop layer by the backing layer; and increases the engagement strength of the loop member. Additionally, the calendered backing layer can have a lower air permeance compared to nonwoven fabric that has not been calendered. As a result, excessively high air permeances that are disadvantageous in the production process can be avoided while maintaining the desired air permeance, which is a result of the contribution obtained by using nonwoven fabric.

In a typical aspect, the backing layer may be calendered to the degree where the backing layer becomes film-like (that is, a state where the smoothness of the nonwoven fabric surface is high and fine printing on the nonwoven fabric surface is possible). The film-like backing layer that has been calendered has a lower air permeance compared to the backing layer prior to the calendering.

However, the present inventor discovered that pliability can be further improved by performing the calendering at a lower roll temperature. By performing the calendering at conditions where the roll temperature is 120° C. or lower, excessively high air permeances that are disadvantageous in the production process can be avoided while maintaining the desired air permeance and, compared to a backing layer that has been subjected to calendering under conditions of about 120° C. to about 180° C., pliability can also be improved. This calendering at a comparatively low temperature is called “low-temperature calendering”.

Thus, in order to impart an appropriate balance of air permeability and pliability, from the perspective of pliability, in a preferable aspect, the roll temperature is 120° C. or lower, 110° C. or lower, or 100° C. or lower and, from the perspective of air permeability, in a preferable aspect, the roll temperature is 30° C. or higher, 50° C. or higher, 65° C. or higher, or 80° C. or higher.

Additionally, from the perspective of air permeability, the pressure of the calendering is 7 MPa or greater or 8 MPa or greater and, from the perspective of pliability, is 15 MPa or less, 13 MPa or less, or 11 MPa or less.

The pliability of the backing layer in a preferable aspect is a degree of pliability as measured by the Cantilever method stipulated in JIS L1096, and the degree of pliability in the machine direction (MD) is 60 mm or less, 50 mm or less, or 40 mm or less. The degree of pliability in the cross direction (CD) is 50 mm or less, 40 mm or less, or 30 mm or less. The degree of pliability measured by a KES (Kawabata's Evaluation System) value is an average value of the machine direction and the cross direction and is 0.00008(8×10⁻⁵) N×cm²/cm or less or 0.00006(6×10⁻⁵) N×cm²/cm or less.

The air permeability of the backing layer in a preferable aspect is a degree of air permeance as measured by the Frazier method stipulated in JIS L1096 and, from the perspective of avoiding excessively high air permeances that are disadvantageous in the production process, the air permeance is 200 cm³/s×cm² or less, 170 cm³/s×cm² or less, 150 cm³/s×cm² or less, 130 cm³/s×cm² or less, or 110 cm³/s×cm² or less; and, for example, in cases where used as a sanitary article, from the perspective of having air permeability to the inside of the sanitary article or to the skin of the wearer, is 10 cm³/s×cm² or greater, 20 cm³/s×cm² or greater, or 30 cm³/s×cm² or greater.

With the loop layer that is combined with the backing layer, which has been subjected to calendering (low-temperature calendering) at a comparatively low temperature (e.g. 120° C. or lower), a short fiber nonwoven fabric that has been fused by the high-temperature air-through processing described above is preferable when intended for adult use because excellent retention of engagement strength when subjected to repeated detaching and attaching will be obtained. On the other hand, when intended for child use, combining a short fiber nonwoven fabric that has not been particularly subjected to high-temperature air-through processing as the loop layer is preferable from the perspectives of pliability, feeling to the skin, and the like. If fused by the high-temperature air-through processing (if the fiber surface is melted and the fixing sites between the fibers are increased), rigidity will increase and the feeling to the skin will take on a stiff feeling. However, with child use products, disposable applications are common and there is not so great a need for repeated detaching and attaching. Therefore, for children that have softer and more sensitive skin than adults, it is preferable that a short fiber nonwoven fabric that has not been particularly subjected to the high-temperature air-through processing (rather, a nonwoven fabric for which the degree of processing is reduced, that is, the processing temperature is lowered as much as possible and a soft feeling is retained) is used in the loop layer.

Thus, a backing layer with high pliability is suitable for child diapers, for example, and by being combined with the fused loop layer, is also suitable for adult-use diapers. The backing layer is not limited to diapers and is suitable for sanitary articles in general. Furthermore, the backing layer that has an appropriate balance of air permeability and pliability can also be used as a short fiber nonwoven fabric in a filter product, a polishing product, or the like. For example, by using the backing layer as a filter product, benefits can be expected such as filtration/absorption of foreign objects being aided due to the blocking of a suitable amount of airflow, and adhesiveness to a filter cartridge being improved due to the high pliability. By applying this filter product to an application of a mask, benefits can be expected such as high microparticle trapping efficiency and flexible conformity to the face.

In a preferred aspect, the thickness of the loop layer is about 0.5 mm or greater and about 20 mm or less. From the perspective of maintaining excellent mechanical strength and obtaining excellent engagement strength and excellent retention of the engagement strength when subjected to repeated detaching and attaching, the thickness of the loop layer is preferably about 0.5 mm or greater, about 1.0 mm or greater, or about 1.5 mm or greater and, from the perspectives of reducing costs and obtaining excellent pliability, is preferably about 20 mm or less, about 10 mm or less, or about 2.0 mm or less.

In an aspect of the present invention, the thickness of the backing layer is about 15 μm or greater and about 100 μm or less. From the perspective of maintaining excellent mechanical strength and obtaining excellent engagement strength, the thickness of the backing layer is about 15 μm or greater, preferably about 20 μm or greater, about 25 μm or greater, or about 35 μm or greater. From the perspectives of reducing costs and obtaining excellent pliability, the thickness is about 100 μm or less, preferably about 85 μm or less, about 70 μm or less, or about 55 μm or less. With a backing layer constituted of short fiber nonwoven fabric (e.g. thermally bonded nonwoven fabric), in cases where the predetermined thickness is attained by calendering, both the reduction of the air permeance of the backing layer to a desired level and the maintaining of the pliability can be achieved.

In the present disclosure, the thicknesses of the loop layer and the backing layer are measured as follows. First, a thickness measurement sample with an area of 10×10 mm is collected from the loop member. Next, when measuring the loop layer, the backing layer is removed from the measurement sample and the thickness of the loop layer, in a state where not bonded or fixed to the backing layer, is measured. When measuring the backing layer, the loop layer is removed from the measurement sample and the thickness of the backing layer, in a state where not bonded or fixed to the loop layer, is measured. This thickness measuring is repeated five times at different locations of the sample, and the average value of the thicknesses resulting from the five measurements is considered as the thickness of the loop layer or the backing layer. A thickness measuring instrument (ABSOLUTE KK-547-055, manufactured by Mitsutoyo, or equivalent measuring instrument) is used to measure the thickness. The thickness of the loop layer or the backing layer is measured by interposing the sample between the cylindrical end face and the base of the measuring instrument and two-seconds thereafter, reading the digitally displayed thickness.

In a preferred aspect, the basis weight of the loop layer is about 12 gsm or greater and about 50 gsm or less. From the perspective of maintaining the mechanical strength of the loop layer and obtaining excellent engagement strength and excellent retention of the engagement strength when subjected to repeated detaching and attaching, the basis weight of the loop layer is preferably about 12 gsm or greater, about 15 gsm or greater, about 20 gsm or greater, or about 25 gsm or greater and, from the perspectives of thinness, reducing costs, and obtaining a pliable loop member, is preferably about 50 gsm or less, about 45 gsm or less, or about 35 gsm or less.

In a preferred aspect, the basis weight of the backing layer is about 8 gsm or greater and about 30 gsm or less. From the perspective of maintaining the mechanical strength of the backing layer, the basis weight is preferably about 8 gsm or greater, about 10 gsm or greater, or about 12 gsm or greater and, from the perspectives of thinness, reducing costs, and obtaining a pliable loop member, is preferably about 30 gsm or less, about 25 gsm or less, about 21 gsm or less, or about 18 gsm or less.

The surface of the print layer forming surface of the backing layer may be subjected to surface treatment (e.g. corona discharge treatment, E-beam treatment). Additionally, the loop layer and/or the backing layer may be subjected to coloring or other treating.

From the perspective of excellently obtaining the advantages of the loop member of the present disclosure, the loop layer and/or the backing layer are typically constituted from a short fiber nonwoven fabric. Additional layers other than short fiber nonwoven fabric may also be included. The loop layer and the backing layer, and the short fiber nonwoven fabric included in the loop layer and the backing layer may each consist of a single layer or may consist of a plurality of layers. Examples of additional layers include adhesive layers, resin films, knitted fabric, woven fabric, paper, laminates of these, and the like. The method for forming the additional layers is not particularly limited, and any conventionally known method such as coating, dry lamination, extrusion lamination, wet lamination, thermal lamination, ultrasonic method, may be used.

In a typical aspect, the loop member further includes a print layer. The print layer may be fixed directly on the backing layer. In the present disclosure, the print layer being fixed directly on the backing layer means that the print layer is disposed on (that is, is in contact with) the backing layer without any other members or layers disposed therebetween, and that the print layer is not substantially peelable from the backing layer while maintaining the form of the print layer. Such a print layer is advantageous because a thin loop member can be obtained through a simple process.

The print layer includes at least an ink layer, and is either a single layer or is a plurality of layers. The print layer may be constituted only of the ink layer or, in addition to the ink layer, may include a base coat and/or a top coat. The base coat and the top coat each contribute to the improvement of the fixibility of the ink layer on the backing layer.

The ink layer and the optional base coat and top coat each may exist as a continuous layer on the backing layer or may be disposed discontinuously; and may be appropriately designed depending on a desired purpose such as, for example, the intended design of the ink layer. Any design may be selected, including characters, drawings, patterns, or the like.

Examples of the material of the ink layer include various types of conventionally known inks, and water-soluble and solvent-based inks may be used. Resins normally used in the art may be used as the resin included in the ink. Examples of such resins include acrylic resins, polyurethane resins, polyamide resins, urea resins, polyester resins, vinyl chloride resins, vinylidene chloride resins, vinyl chloride-vinyl acetate copolymer resins, ethylene-vinyl acetate copolymer resins, olefin resins, chlorinated olefin resins, epoxy resins, petroleum-based resins, cellulose derivative resins, and the like. If the ink layer has excellent toughness, the ink layer will not be prone to becoming defective, even in cases such as where the ink layer is exposed when using an article that is provided with the loop member of the present disclosure. From this perspective, acrylic-based inks, urethane-based inks and the like, for example, are preferable.

From the perspective of the fixibility of the print layer on the backing layer, it is preferable that the ink layer itself be adhesive. Examples of the material for forming the adhesive ink layer include materials obtained by mixing ink with the material used for the adhesive layer described above.

The thickness of each of the ink layer, and optionally the base coat and the top coat, constituting the print layer is not limited, but from the perspectives of the toughness of the print layer and the wear feel (pliability, air permeability, and the like) when used in a sanitary article, is, for example, about 0.5 μm or greater and about 20 μm or less, about 1 μm or greater and about 15 μm or less, or about 2 μm or greater and about 10 μm or less.

In an aspect, the print layer may be adhered to the backing layer by an adhesive. In this case, the fixibility of the print layer is excellent. Examples of the material of the adhesive include adhesive polymers such as acrylic polymers (e.g. SK dyne (an acrylic adhesive commercially available from Soken Chemical & Engineering Co., Ltd.), silicone-based polymers, rubber-based polymers, and the like, and hotmelt-type adhesives such as, for example, Jet-melt™ EC-3748 (commercially available from 3M) and the like. Furthermore, optionally, tackifying resins, crosslinking agents, or other additives may be combined with the adhesive polymers described above.

The thickness of the adhesive is typically from about 5 μm to about 200 μm. The adhesive is formed, for example, by coating the material of the adhesive described above on the surface of the backing layer and, thereafter, drying.

Various production methods are possible for the loop member of the present disclosure. In an illustrated aspect, the loop member can be produced by a method comprising the steps of: preparing the short fiber nonwoven fabric for the loop layer and the short fiber nonwoven fabric for the backing layer; subjecting the short fiber nonwoven fabric of the backing layer to calendering; fusing the short fiber nonwoven fabric of the loop layer; laminating the calendered short fiber nonwoven fabric and the fused short fiber nonwoven fabric for the loop layer; bonding these two layers together so as to obtain a laminated web having the backing layer and the loop layer; and optionally forming the print layer on the backing layer side of the laminated web.

The fabrication of the short fiber nonwoven fabrics, the calendering of the backing layer, and the fusing of the loop layer can, for example, be performed via the method and conditions described above. Next, the calendered short fiber nonwoven fabric and the fused short fiber nonwoven fabric for the loop layer are laminated.

In a typical aspect, the loop layer and the backing layer are bonded to each other by embossing, chemical bonding, water jet, air-through (including high-temperature air-through), or similar processing. FIG. 2 is a drawing illustrating an example of the loop member according to an aspect of the present invention, and depicts a state in which the loop layer and the backing layer are secured by embossing. As illustrated in FIG. 2, with the loop member 1, the backing layer 11 and the loop layer 12 can be bonded to each other by an emboss pattern A. The shape of the emboss pattern formed through the embossing is not particularly limited, and may be rectangular, a wave-shape, or the like. For example, in an illustrated aspect of bonding regions where the loop layer and the backing layer are bonded, in cases where the loop layer and/or the backing layer include carded nonwoven fabric, a pitch of the bonding regions in a fiber direction of the carded nonwoven fabric is preferably disposed so as to be shorter than a pitch of the bonding regions in a direction substantially orthogonal to the fiber direction. Such bonding regions are advantageous because fluffing of the carded nonwoven fabric can be avoided. A structure in which the loop layer is bonded to the calendered backing layer is obtained and, thus, it is possible to securely and strongly fix the loop layer to the backing layer. As such, when peeling the loop member from a male member or the like, problems such as the loop layer peeling from the backing layer, the tearing or destruction of the loop layer, and the like will not easily occur. Conditions of the embossing may be set to, for example, a temperature of about 110° C. to about 180° C. and a pressure in a range from 0 N/m² to about 1000 N/m².

In a case of a loop member in which a backing layer that has been subjected to low-temperature calendering is combined with a loop layer that has not been fused (has not been subjected to high-temperature air-through processing) (e.g. sanitary articles for children are anticipated), the pliability in a preferable aspect is a degree of pliability measured by the Cantilever method stipulated in JIS L1096, and the degree of pliability in the machine direction (MD) is 60 mm or less, 55 mm or less, or 50 mm or less. The degree of pliability in the cross direction (CD) is 50 mm or less, or 45 mm or less.

In a case of a loop member in which a backing layer that has been subjected to low-temperature calendering is combined with a loop layer that has not been fused (has not been subjected to high-temperature air-through processing) (e.g. sanitary articles for children are anticipated), from the perspective of avoiding excessively high air permeances that are disadvantageous in the production process, the air permeance measured by the Frazier method stipulated in JIS L1096, is 100 cm³/s×cm² or less, 95 cm³/s×cm² or less, 90 cm³/s×cm² or less, or 85 cm³/s×cm² or less; and, for example, in cases where used as a sanitary article, from the perspective of having air permeability to the inside of the sanitary article or to the skin of the wearer, is 10 cm³/s×cm² or greater, 20 cm³/s×cm² or greater, or 30 cm³/s×cm² or greater.

Examples of methods for forming the print layer on the backing layer include a method in which a material for forming the print layer (also referred to as a “printing material” in the present disclosure) is coated directly on the backing layer. Additionally, a method can be used in which a print layer that has been pre-formed on a liner is transferred to the backing layer, and then the liner is removed. The transfer method is preferable because the ink of the print layer will not easily penetrate the backing layer and the loop layer.

Specifically, in the transfer method, first, a print layer is formed on a liner. A surface of the liner preferably has releasing properties sufficient to enable the transfer of the print layer to the backing layer. Various types of sheets that are conventionally known as transfer liners can be used as the liner, and examples thereof include silicone-coated kraft paper, silicone-coated polyethylene-coated paper, silicone coated or uncoated polymeric material (e.g., polyethylene, polypropylene, or the like), as well as base materials coated with polymeric release agents such as silicone urea, urethanes, long chain alkyl acrylates, and the like. Examples of suitable commercially available release liners include the product known as POLYSLIK (manufactured by Rexam Release of Oakbrook, Ill.), the product known as EXHERE (manufactured by P.H. Glatfelter Company of Spring Grove, Pa.), and the like. The print layer forming surface of the liner may be subjected to, for example, embossing or the like.

The printing material is applied on the liner described above. In a typical aspect, the ink is applied on the liner via a desired printing method such as, for example, roll coating, gravure coating, curtain coating, spray coating, screen printing, or the like. Alternatively, a top coat, the ink, and a base coat may be applied on the liner, in this order. According to the procedure described above, a transfer sheet in which the print layer is formed on the liner is obtained.

Next, the print layer is transferred from the liner to the backing layer by passing the transfer sheet and the laminated web through a pair of rollers such that the print layer side of the transfer sheet and the backing layer side of the laminated web face each other. Thereby, a loop member in which the print layer is formed on the backing layer can be obtained.

Note that, in cases where the print layer includes the ink layer, and a base coat and/or a top coat, the print layer may be sequentially formed through a plurality of steps. For example, in the case of a print layer including a base coat, the ink layer, and a top coat, the base coat may be formed beforehand on the backing layer, then the ink layer may be transferred, and then the top coat may be formed. The print layer can be directly fixed to the backing layer through this method.

In a preferred aspect, a basis weight of the loop member is about 10 gsm or greater and about 60 gsm or less. From the perspectives of maintaining the mechanical strength of the loop member and obtaining excellent engagement strength, the basis weight is preferably about 10 gsm or greater, about 13 gsm or greater, about 16 gsm or greater, about 20 gsm or greater, about 25 gsm or greater, about 28 gsm or greater, or about 33 gsm or greater and, from the perspectives of thinness, reducing costs, and obtaining a pliable loop member, is preferably about 60 gsm or less, about 50 gsm or less, about 43 gsm or less, or about 37 gsm or less.

In a preferable aspect, a 90-degree peel strength between the loop member and another fastening member with which the loop member is engaged (specifically a hook for a surface fastener) is about 0.2 N/25.4 mm or greater and about 10 N/25.4 mm or less. The 90-degree peel strength is measured in accordance with JTM-1221, using a hook member having 1600 pins/in² (1600DH, manufactured by the 3M Company) as the male member. From the perspective of obtaining excellent engagement strength, the 90-degree peel strength is preferably about 0.2 N/25.4 mm or greater, about 0.3 N/25.4 mm or greater, about 0.4 N/25.4 mm or greater, or about 0.45 N/25.4 mm or greater and, from the perspective of obtaining excellent feeling of use, is preferably about 10 N/25.4 mm or less, about 8 N/25.4 mm or less, about 7 N/25.4 mm or less, or about 6 N/25.4 mm or less.

In a preferable aspect, the 90-degree peel strength between the loop member and another fastening member with which the loop member is engaged (specifically a hook for a surface fastener), at the 20th repetition of 20 repetitions of the 90 degree peeling, is about 0.1 N/25.4 mm or greater and about 5.0 N/25.4 mm or less. The 90-degree peel strength is measured in accordance with JTM-1221, using a hook member having 1600 pins/in² (1600DH, manufactured by the 3M Company) as the male member. From the perspective of obtaining excellent engagement strength, the 90-degree peel strength is preferably about 0.1 N/25.4 mm or greater, about 0.2 N/25.4 mm or greater, about 0.3 N/25.4 mm or greater, or about 0.4 N/25.4 mm or greater and, from the perspective of obtaining excellent feeling of use, is preferably about 5.0 N/25.4 mm or less, about 3.0 N/25.4 mm or less, or about 1.0 N/25.4 mm or less.

In a preferable aspect, a shear strength between the loop member and another fastening member with which the loop member is engaged (specifically a hook for a surface fastener) is about 25 N/20 mm×25.4 mm or greater. The shear strength is a value measured in accordance with JTM-1235, using a hook member having 1600 pins/in² (1600DH, manufactured by the 3M Company) as the male member. From the perspective of obtaining excellent engagement strength, the shear strength is preferably about 25 N/20 mm×25.4 mm or greater or about 30 N/20 mm×25.4 mm or greater. An upper limit of the shear strength is not particularly limited, but, from the perspectives of ease of production and strength of the fastening members, may, for example, be about 100 N/20 mm×25.4 mm or less.

In a preferable aspect, after 20 repetitions of engaging and 90-degree peeling between the loop member and another fastening member with which the loop member is engaged (specifically a hook for a surface fastener) in accordance with JTM-1221, the shear strength is about 5 N/20 mm×25.4 mm or greater and about 100 N/20 mm×25.4 mm or less. The shear strength is a value measured in accordance with JTM-1235, using a hook member having 1600 pins/in² (1600DH, manufactured by the 3M Company) as the male member. From the perspective of obtaining excellent engagement strength, the shear strength is preferably about 5 N/20 mm×25.4 mm or greater, about 9 N/20 mm×25.4 mm or greater, or about 14 N/20 mm×25.4 mm or greater. An upper limit of the shear strength is not particularly limited, but, from the perspectives of ease of production and strength of the fastening members, may, for example, be about 100 N/20 mm×25.4 mm or less.

In a preferable aspect, tensile strength of the loop member at 5% elongation is controlled to a predetermined range, depending on the purpose. If the tensile strength of the loop member is excessively high, the loop member will tend to be rigid. On the other hand, if the tensile strength of the loop member is excessively low, there is a possibility that disadvantages may occur such as the loop member becoming elongated in the machine direction (cases where the tensile strength in the machine direction is excessively low) when producing sanitary articles (diapers or the like), or the loop layer becoming elongated at a time of use of a sanitary article (diaper or the like) provided with the loop member due to shearing with the hook with which the loop member is combined (cases where the tensile strength in the cross direction is excessively low). Note that in the present disclosure, the machine direction of the loop member refers to the machine direction of the loop member at the time of production. The machine direction of the loop member at the time of production normally matches the machine directions of the loop layer and the backing layer at the time of production. Additionally, the cross direction refers to a direction orthogonal to (that is, that forms a 90 degree angle with) the machine direction. From the perspectives described above, the tensile strength in the machine direction preferably may be about 7 N/25.4 mm or greater, about 10 N/25.4 mm or greater, or about 12 N/25.4 mm or greater, and preferably may be about 200 N/25.4 mm or less, about 100 N/25.4 mm or less, or about 50 N/25.4 mm or less. Additionally, the tensile strength in the cross direction preferably may be about 2.5 N/25.4 mm or greater, about 3 N/25.4 mm or greater, or about 3.5 N/25.4 mm or greater, and preferably may be about 50 N/25.4 mm or less, about 30 N/25.4 mm or less, or about 20 N/25.4 mm or less.

The loop member of the present disclosure can be used for various articles such as, for example, for the fixing of various types of applicable subjects to floors, walls, clothing, cleaning members, automobile interior materials, and the like. The loop member of the present disclosure, due to the configuration thereof, can have excellent pliability and air permeability, excellent engagement strength, and excellent retention of the engagement strength after being subjected to repeated detaching and attaching. As such, the loop member of the present disclosure is particularly suitable as a loop member for surface fastener to be attached to an adult diaper.

In another aspect of the present invention, an adult diaper is provided that includes a surface fastener including the loop member according to the aspect of the present invention described above.

Examples of sanitary articles include child and adult diapers, napkins for sanitary and other uses, and the like, but the loop member of the present disclosure has excellent retention of engagement strength when subjected to repeated detaching and attaching and, thus, is particularly suited to adult diapers that are frequently attached and detached. In a typical aspect, the loop member of the present disclosure can be combined with the loop member of the present disclosure or another desired fastening member, respectively, and be used as a surface fastener of a sanitary article, preferably an adult diaper.

In a preferred aspect, the sanitary article has excellent air permeability. More specifically, from the perspective of imparting excellent wear feel to the sanitary article, air permeance of the sanitary article, measured by the Gurley method, is preferably about 5 seconds or less. The air permeance is more preferably about 3 seconds or less and even more preferably about 1 second or less. A lower limit is not particularly limited but, in an aspect, is about 0.1 seconds or greater.

A production method of the sanitary article is not particularly limited, and an example of a method is described below. Any conventionally known constituent can be used in addition to the loop member in the sanitary article, and specific description thereof is omitted herein. In the sanitary article, the method for attaching the loop member to the application section may be any conventionally known method. The print layer forming surface of the loop member is joined to the application section by a conventionally known joining method (gluing, heat fusing, bonding by ultrasonic machining or the like, mechanical fixing by sewing, stapling, or the like). For fastening by means of gluing, known adhesives such as rubber based adhesives such as SIS and SBS, acryl based adhesives, silicone based adhesives, EVA based adhesives, and the like may be suitably selected as required, but the adhesive is not limited to those resins.

An aspect of the present invention provides a loop member and includes the following:

a loop member for surface fastener, including a loop layer and a backing layer; wherein

the loop layer includes fused short fiber nonwoven fabric;

the backing layer includes calendered short fiber nonwoven fabric;

a ratio of an average fineness of the fiber in the loop layer to an average fineness of the fiber in the backing layer (average fineness of fiber in the loop layer/average fineness of fiber in the backing layer) is from 1.5 to 30; and

a thickness of the backing layer is from 15 μm to 100 μm.

Additionally, another aspect provides an adult diaper including this loop member.

According to these aspects, a loop member for surface fastener and an adult diaper can be provided that have excellent air permeability, pliability, engagement strength, retention of engagement strength when subjected to repeated detaching and attaching, and print characteristics, and which can be produced at a low cost.

### EXAMPLES

The present invention will now be described in further detail using examples, but the present invention is not limited to these examples. Working Examples 1 to 7 are descriptions of examples of loop members provided with loop layers that have been subjected to high-temperature air-through processing. There are suitable for the loop member of, for example, an adult diaper. On the other hand, the Working Examples B1 to B6 are description of examples of loop members provided with backing layers that have been subjected to low-temperature calendering and loop layers that have not been subjected to high-temperature air-through processing (Working Examples A1 to A5 are descriptions of examples where only the backing layer has been subjected to low-temperature calendering). These are suitable for the loop member of, for example, a child diaper.

### Fabrication of Loop Member Including Fused Loop Layer

### Working Examples 1 to 7 and Comparative Example 1

### Short Fiber Nonwoven Fabric for the Loop Layer

The following carded nonwoven fabrics were used. The melting point of the sheath was about 115° C. and the melting point of the core was about 163° C.

Product: 6.6 ESC cure repeat PE 1185, polyethylene (sheath)/polypropylene (core) two-component fiber,

average fineness: 6 denier, fiber length: 40 mm, commercially available from Fibervisions.

Product: SESC4013, polyethylene (sheath)/polypropylene (core) two-component fiber, average fineness: 4 denier, fiber length: 40 mm, commercially available from Fibervisions.

Product: ESC225SDGK, average fineness: 2 denier, polyethylene (sheath)/polypropylene (core) two-component fiber, fiber length: 40 mm, commercially available from Fibervisions.

Product: ETC212C, average fineness: 12 denier, polyethylene (sheath)/polyethylene terephthalate (core) two-component fiber, fiber length: 40 mm, commercially available from Fibervisions.

### Fusing of the Loop Layer

In the oven described below and under the following conditions, the short fiber nonwoven fabric was subjected to high-temperature air-through processing. Thus, a short fiber nonwoven fabric for the loop layer was obtained.

Oven: STRAHM HiPer™ Therm System (manufactured by Strahm Textile Systems AG)

Line speed: As shown in Tables 1 and 2

Air-through temperature: As shown in Tables 1 and 2

Air blow amount*: As shown in Tables 1 and 2

*Proportion of 100% of the maximum blow amount of the oven

Additionally, a carded nonwoven fabric constituted of polyethylene (sheath)/polypropylene (core) two-component fibers (Product: 1.7 ESC cure repeat PE 1185 or SESC4014, average fineness: 1.5 denier, fiber length: 40 mm, commercially available from Fibervisions) was passed through a 150° C. thermal point bonding roller and, thereafter, was subjected to 100% calendering. Thus, a short fiber nonwoven fabric for the backing layer (basis weight: 15 gsm, fineness: 1.5 denier) was obtained.

The short fiber nonwoven fabric for the loop layer and the short fiber nonwoven fabric for the backing layer obtained as described above were stacked and laminated by pattern embossing at a temperature of 135° C. and a nip pressure of 80 kg (8 MPa). Thus, the loop member was obtained.

### Comparative Example 2

KLL GKLL: Product: CLP-06603, commercially available from the 3M Company.

### Characteristics Evaluation of Loop Member Including Fused Loop Layer

### 1. 90-Degree Peel Strength

The 90-degree peel strength was measured in accordance with JTM-1221, using a hook member having 1600 pins/in² (1600DH, manufactured by the 3M Company) as the male member. Engaging and 90-degree peeling of the loop member and the male member was repeated 20 times and the peel strengths at the 1st repetition and at the 20th repetition were recorded.

### 2. Shear Strength

The shear strength was measured in accordance with JTM-1235, using a hook member having 1600 pins/in² (1600DH, manufactured by the 3M Company) as the male member. The shear strength measured after engaging the loop member and the male member was set as the 1st shear strength and in accordance with JTM-1221 and via the method described above in 1., engaging and 90-degree peeling of the loop member and the male member was repeated 20 times. Then, the shear strength measured after engaging the loop member and the male member was set as the shear strength at the 20th repetition.

### 3. Tensile Strength

The tensile strength of the loop layer was measured via the method described below, using a Tensilon universal testing machine (RTG-1225, manufactured by A&D Company, Ltd.).

A laser cutter was used to cut and fabricate a sample with a length of at least 100 mm and a width of 50 mm from the loop layer.

The Tensilon settings were as follows:

Chuck interval: 100 mm

Pulling speed: 300 mm/min

The sample was attached to the chuck and the tensile strength at break was measured.

### 4. Loop Layer Surface Condition

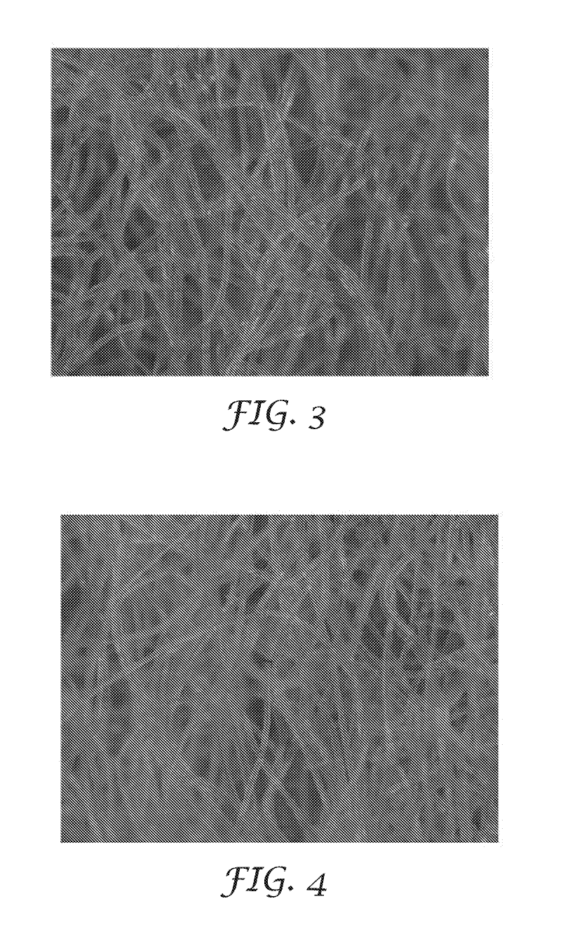

The surface states of the loop layers of the loop members fabricated in Working Example 1 and Comparative Example 1 were observed using an optical microscope. FIG. 3 is a figure showing a surface state image of the loop layer of Working Example 1. FIG. 4 is a figure showing a surface state image of the loop layer of Comparative Example 1. FIGS. 3 and 4 are each figures that were imaged at an enlargement factor of 175-times.

TABLE 1

| | | | Working | Working | Working | Working | Comparative | Comparative |
|---|---|---|---|---|---|---|---|---|
| | | | Example 1 | Example 2 | Example 3 | Example 4 | Example 1 | Example 2 |
| Loop layer | Fineness (denier) | | 6 | 4 | 2 | 12 | 4 | — |
| | Fiber material | | PE/PP | PE/PP | PE/PP | PE/PET | PE/PP | PET |
| | | | (sheath/core) | (sheath/core) | (sheath/core) | (sheath/core) | (sheath/core) | (KLL) |
| | Basis weight (g/m²) | | 30 | 30 | 30 | 30 | 30 | — |
| | Line speed (mi/min) | | 60 | 60 | 60 | 60 | 60 | — |
| | Air-through temperature | | 142 | 142 | 142 | 142 | 131 | — |
| | (° C.) | | | | | | | |
| | Air blow amount (%) | | 30 | 30 | 30 | 30 | 23 | — |
| | Tensile strength at break | | 31 | 42 | NA | 21 | 20 | — |
| | (N/50 mm) | | | | | | | |
| | Thickness (mm) | | 1.38 | 1.03 | NA | 1.6 | 1.27 | — |
| Backing | Fineness (denier) | | 1.5 | 1.5 | 1.5 | 1.5 | 1.5 | — |
| layer | Fiber material | | PE/PP | PE/PP | PE/PP | PE/PP | PE/PP | — |
| | Basis weight (g/m²) | | 15 | 15 | 15 | 15 | 15 | — |
| Rating | Peel strength | 1st | 3.0 | 2.5 | 0.6 | 2.5 | 3.7 | 2.0 |
| | (JTM1221) | repetition | | | | | | |
| | (N/25.4 mm) | 20th | 0.5 | 0.45 | 0.47 | 0.2 | NA | 0.45 |
| | | repetition | | | | | | |
| | Shear strength | 1st | 48 | 42 | 39 | 35 | 45 | 48 |
| | (JTM1235) | repetition | | | | | | |
| | (N/20 mm × | 20th | 15 | 16 | 14 | 9 | 0 | 17 |
| | 25.4 mm) | repetition | | | | | | |

TABLE 2

| | | | Working | Working | Working |
|---|---|---|---|---|---|
| | | | Examples 5 | Examples 6 | Examples 7 |
| Loop | Fineness (denier) | | 6 | 4 | 2 |
| layer | Fiber material | | PE/PP | PE/PP | PE/PP |
| | | | (sheath/core) | (sheath/core) | (sheath/core) |
| | Basis weight (g/m²) | | 30 | 30 | 30 |
| | Line speed (mi/min) | | 60 | 60 | 60 |
| | Air-through temperature (° C.) | | 146 | 146 | 146 |
| | Air blow amount (%) | | 30 | 30 | 30 |
| Backing | Fineness (denier) | | 1.5 | 1.5 | 1.5 |
| layer | Fiber material | | PE/PP | PE/PP | PE/PP |
| | Basis weight (g/m²) | | 15 | 15 | 15 |
| Rating | Peel strength | 1st | 0.98 | 0.25 | 0.2 |
| | (JTM1221) | repetition | | | |
| | (N/25.4 mm) | 20th | NA | NA | NA |
| | | repetition | | | |
| | Shear strength | 1st | 32 | 7 | 19 |
| | (JTM1235) | repetition | | | |
| | (N/20 mm × 25.4 mm) | 20th | NA | NA | NA |
| | | repetition | | | |

Excellent peel strength and shear strength were displayed by the loop members according to each of the Working Examples. With Working Examples 1 to 4, excellent peel strength and shear strength (that is, excellent retention of peel strength and shear strength) were displayed even after 20 repetitions of detaching and attaching. The retention of the peel strength and the shear strength for each of the Working Examples was not inferior to the knit loop of Comparative Example 2. On the other hand, with Comparative Example 1 for which a nonwoven fabric loop member was used, in the peel testing, retention of the lamination of the loop layer failed at the 10th to 15th repetition, or the shear strength decreased dramatically due to the repeated detaching and attaching. In Comparative Example 1, the loop layer was formed under low-temperature air-through conditions and, consequently, the loop layer was substantially free of fusing.

### Fabrication of Backing Layer Subjected to Low-Temperature Calendering

### Working Examples A1 to A5 and Comparative Examples A1 to A5

### Short Fiber Nonwoven Fabric for the Backing Layer

The following carded nonwoven fabrics were used for the backing layer. The melting point of the sheath was about 115° C. and the melting point of the core was about 163° C.

Average fineness: 1.5 denier, polyethylene (sheath)/polypropylene (core) two-component fiber, fiber length: 40 mm, basis weight: 15 gsm, manufactured by Fibervisions

Average fineness: 1.2 denier, polyethylene (sheath)/polypropylene (core) two-component fiber, fiber length: 40 mm, basis weight: 15 gsm, commercially available from Fibervisions (product: ESC112)

### Calendering of the Backing Layer

Short fiber nonwoven fabric was passed through thermal point bonding rollers and temporarily fixed and, thereafter, was subjected to 100% calendering (calender processing). Thus, the short fiber nonwoven fabric for the backing layer was obtained. The fineness of this short fiber nonwoven fabric and the conditions of the calendering are as shown in Table 3. The calendering performed at comparatively low temperatures such as that in Working Examples A1 to A5 is referred to as “low-temperature calendering”.

### Fabrication of Loop Member Including Backing Layer Subjected to Low-Temperature Calendering

### Working Examples B1 to B6 and Comparative Examples B1 and B2

### Short Fiber Nonwoven Fabric for the Loop Layer

Polyethylene (sheath)/polypropylene (core) two-component fiber, average fineness: 4 denier, fiber length: 40 mm, manufactured by Fibervisions.

### Creation of Loop Member

The short fiber nonwoven fabric for the loop layer described above and the short fiber nonwoven fabric for the backing layer that has been subjected to low-temperature calendering were stacked and laminated by pattern embossing at a temperature of 135° C. and a nip pressure of 5 MPa. Thus, the loop member was obtained.

### Characteristics Evaluation of Backing Layer Subjected to Low-Temperature Calendering and Loop Member Including Same

### 1. Shear Strength and Tensile Strength

The shear strength and the tensile strength were evaluated in the same manner as described above in “Characteristics Evaluation of Loop Member Including Fused Loop Layer”.

### 2. Degree of Pliability According to the Cantilever Method of JIS L1096

A 25 mm×250 mm test piece was collected from the sample. A first edge of the test piece was aligned with and placed on the front end of the platform of a Cantilever-type testing machine. The 0 point of the steel ruler was adjusted to the mark D and, in this state, the steel ruler was placed on the test piece. The steel ruler and the test piece were pressed gently together at a constant speed in the direction of the inclined face. The steel ruler was moved and the test piece was allowed to rest for eight seconds and, then, the length of the protruding test piece was read from the steel ruler.

### 3. Degree of Pliability According to KES

A KESFB2-S, manufactured by Kato Tech was used as the testing machine. A test piece was collected from the sample and set in the testing machine. Thus, the flexural rigidity (gf×cm²/cm) was obtained. Note that 1 gf×cm²/cm is equivalent to 0.0098 N×cm²/cm, and both values are recorded in Table 3. Also, in Table 3, “5.4E-05”, means “5.4×10⁻⁵”, for example.

### 4. Air Permeance According to the Frazier Method of JIS L1096

A machine manufactured by Toyo Seiki Seisaku-Sho. Ltd. was used as the Frazier-type testing machine. A 150 mm×150 mm test piece was collected from the sample and the test piece was attached to a first end of the cylinder of the testing machine. Then, the intake fan and air ports were adjusted using the variable resistor so that the inclined barometer read 125 Pa and pressure shown on the vertical barometer at that time was measured. Using the conversion tables provided with the testing machine, the air flow (cm³/sec×cm²) passing through the test piece was calculated from the measured pressure and the type of air ports.

TABLE 3

| | | | Working | Working | Working | Working | Working |
|---|---|---|---|---|---|---|---|
| | | | Example A1 | Example A2 | Example A3 | Example A4 | Example A5 |
| Backing layer | Fiber | denier | 1.5 | 1.5 | 1.2 | 1.2 | 1.2 |
| (short fiber | Basis weight | g/m² | 18 | 15 | 18 | 18 | 15 |
| nonwoven | | ° C. | 102 | 102 | 97 | 108 | 108 |
| fabric) | Calendering | MPa | 8 | 8 | 8 | 8 | 8 |
| | | Line speed | 30 | 30 | 30 | 60 | 60 |
| | | mi/min | | | | | |
| | Thickness | mm | 0.0417 | 0.0376 | 0.0347 | 0.0338 | 0.0326 |
| | Cantilever | mm | 31 | 32 | 34 | 34 | 36 |
| | (MD) | | | | | | |
| | Cantilever | mm | 24 | 23 | 23 | 20 | 23 |
| | (CD) | | | | | | |
| | KES | gf * cm²/cm | 0.0055 | 0.0015 | 0.0031 | 0.0023 | 0.0045 |
| | | N * cm²/cm | 5.4E−05 | 1.5E−05 | 3.0E−05 | 2.3E−05 | 4.4E−05 |
| | Frazier method | cm³/cm² * sec | 91.1 | 124.9 | 101.8 | 125 | 142.9 |
| | | | Comparative | Comparative | Comparative | Comparative | Comparative |
| | | | Example A1 | Example A2 | Example A3 | Example A4 | Example A5 |
| Backing layer | Fiber | denier | 1.5 | 1.5 | 1.2 | 1.2 | 1.5 |
| (short fiber | Basis weight | g/m² | 18 | 15 | 18 | 15 | 15 |
| nonwoven | | ° C. | — | — | — | — | 130 |
| fabric) | Calendering | MPa | — | — | — | — | 7 |
| | | Line speed | | | | | 60 |
| | | mi/min | | | | | |
| | Thickness | mm | 0.111 | 0.089 | 0.114 | 0.089 | 0.0307 |
| | Cantilever | mm | 37 | 31 | 41 | 38 | 44 |
| | (MD) | | | | | | |
| | Cantilever | mm | 22 | 20 | 22 | 18 | 32 |
| | (CD) | | | | | | |
| | KES | gf * cm²/cm | | 0.0050 | | | 0.0091 |
| | | N * cm²/cm | | 4.9E−05 | | | 8.9E−05 |
| | Frazier method | cm³/cm² * sec | 366 | 450 | 289 | 390 | 132.9 |

As shown in Table 3, with Working Examples A1 to A5, the numerical values of the Frazier method were low compared to Comparative Examples A1 to A5 in which the calendering was not performed. That is, with Working Examples A1 to A5, the air permeance is appropriately decreased. On the other hand, compared to Comparative Example A5 in which the calendering was performed at 130° C., both the Cantilever values and the KES values were low, meaning that the degree of pliability had increased (the nonwoven fabric had become soft)

TABLE 4

| | | | | | Working | Working | Working | Working |
|---|---|---|---|---|---|---|---|---|
| | | | Working | Working | Example | Example | Example | Example |
| | | | Example B1 | Example B2 | B3 | B4 | B5 | B6 |
| Backing | Fiber | denier | 1.5 | 1.5 | 1.2 | 1.2 | 1.2 | 1.2 |
| layer | Basis weight | g/m² | 18 | 15 | 18 | 18 | 15 | 18 |
| | Calendering | ° C. | 102 | 102 | 97 | 108 | 108 | 85 |
| | | MPa | 8 | 8 | 8 | 8 | 8 | 7 |
| | | Line speed | 30 | 30 | 30 | 60 | 60 | 30 |
| | | mi/min | | | | | | |
| Loop layer | Fiber | denier | 4 | 4 | 4 | 4 | 4 | 4 |
| | Basis weight | g/m² | 20 | 20 | 20 | 20 | 20 | 20 |
| Loop | 135dPeel wo load (500 g) | N/25 mm | 0.6 | 0.6 | 0.8 | 0.8 | 0.6 | 0.6 |
| member | 135dPeel w load (500 g) | N/25 mm | 1.0 | 1.0 | 0.9 | 1.0 | 1.1 | 1.0 |
| (=backing | Shear | N | 40.3 | 41.7 | 45.1 | 46.3 | 47.2 | 44 |
| layer + loop | Cantilever (MD) | mm | 53.8 | 47.3 | 49.0 | 50.0 | 51.8 | 56 |
| layer) | Cantilever (CD) | mm | 43.0 | 46.3 | 50.0 | 48.8 | 49.8 | 46 |
| | Frazier method | cm³/cm² * sec | 53 | 69 | 83 | 69 | 87 | 100 |

| | | | Comparative | Comparative |
|---|---|---|---|---|
| | | | Example B1 | Example B2 |
| Backing | Fiber | denier | 1.5 | 1.5 |
| layer | Basis weight | g/m² | 15 | 15 |
| | Calendering | ° C. | 130 | — |
| | | MPa | 7 | — |
| | | Line speed | 60 | — |
| | | mi/min | | |
| Loop layer | Fiber | denier | 4 | 4 |
| | Basis weight | g/m² | 20 | 20 |
| Loop | 135dPeel wo load (500 g) | N/25 mm | 1 | 1 |
| member | 135dPeel w load (500 g) | N/25 mm | 1.2 | 1.2 |
| (=backing | Shear | N | 42 | 45 |
| layer + loop | Cantilever (MD) | mm | 65 | 48 |
| layer) | Cantilever (CD) | mm | 47 | 40 |
| | Frazier method | cm³/cm² * sec | 80 | 170 |

As shown in Table 4, with Working Examples B1 to B6, the numerical values of the Frazier method were low compared to Comparative Example B2 where included the backing layer that was not subjected to the calendering. That is, with Working Examples B1 to B6, the air permeance is appropriately decreased. On the other hand, compared to Comparative Example B1 that included the base material layer that was calendered at 130° C., the Cantilever values (particularly in the machine direction) were low, meaning that the degree of pliability had increased (the nonwoven fabric had become soft).

Description has been given of examples in which a loop layer that has been subjected to high-temperature air-through processing is combined with a backing layer that has been subjected to calendering (Working Examples 1 to 7, suitable as a loop member of an adult diaper, for example); and examples in which a loop layer that has not been subjected to high-temperature air-through processing is combined with a backing layer that has been subjected to low-temperature calendering (Working Examples B1 to B6, suitable as a loop member of a child diaper, for example). Other than these, examples are possible in which a loop layer that has been subjected to high-temperature air-through processing is combined with a backing layer that has been subjected to low-temperature calendering. According to this high-temperature air-through processed loop layer+low-temperature calendered backing layer combination, a loop member is provided that includes a loop layer with high durability, and for which air permeability and pliability are appropriately balanced.

Additionally, it is also possible to subject the loop layer side to high-temperature air-through processing after combining the loop layer not subjected to high-temperature air-through processing with the low-temperature calendered backing layer. However, in this case, the characteristics obtained from the low-temperature calendering may be displayed easily due to the high-temperature air-through passing through and contacting the backing layer. Thus, from the perspective of making use of the differences in the characteristics of each of the layers, the aforementioned configuration is preferable.

Additionally, the materials, temperatures, pressures, and the like may be varied within the spirit and scope of the invention.

### INDUSTRIAL APPLICABILITY

The loop member of the present disclosure can be advantageously used in sanitary articles, for example, and particularly in adult and child diapers.

1. Loop component for mechanical fastener including top layer comprising short-fiber nonwoven and substrate layer comprising short-fiber nonwoven, wherein
ratio [average fineness (denier) of the top layer] against [average fineness (denier) of the substrate layer] is 1.5-30,
softness of the substrate is less than 60 mm in MD direction and less than 50 mm in CD direction by 45 degree cantilever method, and
air permeability of the substrate is 10 to 100 cm3/sec*cm2 by Frajour type method.

2. The loop component described in claim 1, the substrate layer is compressed with temperature that is lower than the melting point of the surface of the short fiber in the substrate layer.

3. The loop component described in claim 1, the substrate layer is compressed with temperature that is lower than 120 degrees Celsius.

4. The loop component described in claim 1, the substrate layer is compressed with pressure of more than 7 MPa.

5. The loop component of described in claim 1, 90 degree peel strength is between 0.2N/25.4 mm and 5.0N/25.4 mm against hook component, when loop and hook components are detached (20th detached) after repeatedly attached/detached 20 times.

6. The loop component described in claim 1, the loop layer includes fused short-fiber nonwoven.

7. The loop component described in claim 6, the short-fiber nonwoven in the loop layer includes core having 1st melting point and sheath having 2nd melting point that is lower than the 1st melting point, wherein;
the loop layer is fused with temperature between the 1st and 2nd melting points.

8. The loop component described in claim 6, the short-fiber nonwoven in the loop layer includes polypropylene core and polyethylene sheath structure, wherein; the loop layer is fused with temperature between 135 and 145 degrees Celsius.

9. The loop component described in claim 1, further comprises printing layer on the substrate layer.

10. Sanitary article including the loop component described in claim 1.

11. Short-fiber nonwoven, wherein;
softness of the substrate is less than 40 mm in MD direction and less than 30 mm in CD direction by 45 degree cantilever method, and air permeability of the substrate is less than 150 cm3/sec*cm2 by Frajour type method.

12. Short-fiber nonwoven, wherein;
softness of the substrate is less 0.00008N*cm2/cm by KES method, and air permeability of the substrate is less than 150 cm3/sec*cm2 by Frajour type method.
